(19) United States
(12) Patent Application Publication
(10) Pub. No.: US 2026/0114719 A1
(43) Pub. Date: Apr. 30, 2026

(54) DEVICES, SYSTEMS, AND METHODS TO SUPPLY FLUIDS TO AN ENDOSCOPE

(71) Applicant: Boston Scientific Scimed, Inc., Maple Grove, MN (US)

(72) Inventors: Ryan Vincent William Pollock, Sterling, MA (US); Scott Edward Corbeil, Litchfield, NH (US)

(73) Assignee: Boston Scientific Scimed, Inc., Maple Grove, MN (US)

(21) Appl. No.: 19/005,356

(22) Filed: Dec. 30, 2024

Related U.S. Application Data
(60) Provisional application No. 63/616,994, filed on Jan. 2, 2024.

(51) Int. Cl. A61B 1/015; A61B 1/12

(52) U.S. Cl. CPC A61B 1/015; A61B 1/126

(57) ABSTRACT

Devices, systems, and methods to supply fluids to an endoscope. An illustrative valve may include a valve body defining a first port configured to receive fluid from a container, a second port configured to be in fluid communication with the first port via a first flow path, a third port configured to be in fluid communication with the first port via a second flow path and a passage extending between the first port, the second port, and the third port. The valve may include a plunger configured to move in the passage between a first position at which the first flow path is open and the second flow path is closed and a second position at which the first flow path and the second flow path are open. The valve may include a biasing mechanism configured to bias the plunger to the first position.

### CROSS-REFERENCE TO RELATED APPLICATIONS

This application claims the benefit of U.S. Provisional Patent Application Ser. No. 63/616,994 filed on Jan. 2, 2024, the disclosure of which is incorporated herein by reference.

### FIELD

This disclosure relates generally to medical fluid containers and methods, and particularly to a container, a valve, and tube sets to supply fluid and/or gas to an endoscope.

### BACKGROUND

Conventionally, endoscope devices have been widely used for performing diagnostic and/or therapeutic treatments. Such endoscope devices sometimes include a fluid capability, or the like, configured to feed fluid to the end of the endoscope for insufflating the inside of the patient at the target site. Lens wash provides a liquid such as sterilized water at relatively high pressure to spray across and clear the camera lens of debris. The water source for lens wash and irrigation typically has included one or more fluid reservoirs with tubing and cap assemblies that creates the plumbing circuit in connection with the endoscope channels and valving to accomplish the gas and water functions described. Such tubing and cap assemblies are available in various configurations, which typically involve a water bottle, a cap fitted for the specific bottle, and an array of tubing that is extendable through openings in the cap. The tubing typically is arranged to accommodate a specific configuration of endoscope fittings and valving.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

A valve for fluidly connecting a lumen in an endoscope to a fluid reservoir is disclosed. The valve comprises a valve body defining: a first port configured to receive fluid from a container; a second port configured to be in fluid communication with the first port via a first flow path; a third port configured to be in fluid communication with the first port via a second flow path; a passage extending between the first port, the second port, and the third port; a plunger configured to move in the passage between: a first position at which the first flow path is open and the second flow path is closed; and a second position at which the first flow path and the second flow path are open; and a biasing mechanism configured to bias the plunger to the first position.

Alternatively or additionally to any of the embodiments above, the first port includes a spike port adaptor that is configured to be inserted into a port of the container.

Alternatively or additionally to any of the embodiments above, a first end of the plunger extends a distance outside of the valve body and forms a lens wash button.

Alternatively or additionally to any of the embodiments above, the plunger is configured to move from the first position to the second position responsive to actuation of the lens wash button.

Alternatively or additionally to any of the embodiments above, the plunger is configured to move from the second position to the first position responsive to release of the lens wash button.

Alternatively or additionally to any of the embodiments above, the biasing mechanism is a spring.

Alternatively or additionally to any of the embodiments above, the container is a flexible bag.

Alternatively or additionally to any of the embodiments above, the plunger is configured as a double-piston plunger including a first seal and a second seal, and wherein the double-piston plunger includes a center portion with a diameter that is less than a diameter at the first seal and is less than a diameter at the second seal.

Alternatively or additionally to any of the embodiments above, the first port is located on a first side of the valve body and wherein the second port and the third port are located on a second side of the valve body.

Alternatively or additionally to any of the embodiments above, the first side is opposite the second side.

Alternatively or additionally to any of the embodiments above, the second port or the third port is coupled to a lumen of lens wash tubing

Alternatively or additionally to any of the embodiments above, the other of the second port or the third port is coupled to a lumen of irrigation supply tubing.

A container, valve, and tube set arranged and configured to couple to an endoscope for use in an endoscopic procedure is disclosed. The container, valve, and tube set comprising: a container configured to contain a fluid, the container having a top portion and a bottom portion, the container having a port in fluid communication with the bottom portion; fluid supply tubing including a first end, a second end, and a lumen extending therethrough, wherein the first end is in fluid communication with the port of the container and the second end is positioned external to the container; a valve including a valve body defining: a first port configured to receive fluid from a container via the fluid supply tubing, wherein the first port includes a spike port adaptor that is configured to be inserted into the port of the container; a second port configured to be in fluid communication with the first port via a first flow path; a third port configured to be in fluid communication with the first port via a second flow path; a passage extending between the first port, the second port, and the third port; a plunger configured to move in the passage between: a first position at which the first flow path is open and the second flow path is closed; and a second position at which the first flow path and the second flow path are open; and a biasing mechanism configured to bias the plunger to the first position; lens wash supply tubing including a first end, a second end, and a lumen extending therethrough, wherein the first end of the lens wash supply tubing is configured to be in fluid communication with the bottom portion of the container via the second flow path when the plunger is in the second position and the second end of the lens wash supply tubing is positioned external to the container and the valve; and irrigation supply tubing having a first end, a second end, and a lumen extending therethrough, wherein the first end of the irrigation supply tubing is configured to be in fluid communication with the bottom portion of the container via first flow path, and the second end of the irrigation supply tubing is positioned external to the container and the valve.

Alternatively or additionally to any of the embodiments above, the valve is configured to continually provide fluid to the irrigation supply tubing when the plunger is in either the first position or the second position.

Alternatively or additionally to any of the embodiments above, further comprising a gas supply tubing including a first end, a second end, and a third extending therethrough, wherein the lumen of the gas supply tubing is in operative communication with the top portion of the container and the second end of the gas supply tubing is positioned external to the container.

Alternatively or additionally to any of the embodiments above, further comprising: a first seal located along an interface between a first end of the plunger and the valve body; and a second seal located along an interface between a second end of the plunger and the valve body.

Alternatively or additionally to any of the embodiments above, wherein the second seal is configured to prevent pressurized gas from backflowing along the second flow path into the container through the first port when the plunger is in the first position.

A container, valve, and tube set arranged and configured to couple to an endoscope for use in an endoscopic procedure is disclosed. The container, valve, and tube set comprising a container configured to contain a fluid, the container having a top portion and a bottom portion, the container having a port in fluid communication with the bottom portion; a valve including: a valve body defining: a first port configured to receive fluid from a container via fluid supply tubing, wherein the first port includes a spike port adaptor that is configured to be inserted into the port of the container; a second port configured to be in fluid communication with the first port via a first flow path; a third port configured to be in fluid communication with the first port via a second flow path; a passage extending between the first port, the second port, and the third port; a plunger configured to move in the passage between: a first position at which the first flow path is open and the second flow path is closed; and a second position at which the first flow path and the second flow path are open; and a biasing mechanism configured to bias the plunger to the first position; and lens wash supply tubing including a first end, a second end, and a lumen extending therethrough, wherein the first end of the lens wash supply tubing is configured to be in fluid communication with the bottom portion of the container via the second flow path when the plunger is in the second position and the second end of the lens wash supply tubing is positioned external to the container and the valve; gas supply tubing including a first end, a second end, and a lumen extending therethrough, wherein the lumen of the gas supply tubing is in operative communication with the top portion and the second end of the gas supply tubing is positioned external to the container; and irrigation supply tubing having a first end, a second end, and a lumen extending therethrough, wherein the first end of the irrigation supply tubing is configured to be permanently in fluid communication with the bottom portion of the container via first flow path to continually supply liquid to the endoscope, and the second end of the irrigation supply tubing is positioned external to the container and the valve.

Alternatively or additionally to any of the embodiments above, the plunger further comprises a dual piston having a first seal at a first end of the dual piston and a second seal at a second end of the dual piston that is opposite the first end.

Alternatively or additionally to any of the embodiments above, the first port includes a spike port adaptor that is configured to be directly inserted into the port of the container.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3A depicts an endoscope system with endoscope, light source, water reservoir, and tubing assembly for hybrid air, lens wash and irrigation fluid delivery;

FIG. 3B depicts the endoscope system of FIG. 3A, wherein the system is activated to deliver air to a patient through the patient end of the endoscope;

FIG. 3C depicts the endoscope system of FIG. 3A, wherein the system is activated to deliver lens wash fluid through the patient end of the endoscope;

FIG. 3D depicts the endoscope system of FIG. 3A, wherein the system is activated to deliver irrigation fluid through the patient end of the endoscope;

FIG. 4 depicts a hybrid endoscope system including a video processing unit, connector portion, peristaltic irrigation pump, water reservoir and top, coaxial gas and lens wash supply tubing, upstream and downstream irrigation supply tubing, and alternative gas supply tubing;

FIG. 5 depicts an illustrative endoscope system having a fluid supply system and a valve;

FIG. 5A depicts another illustrative endoscope system having a fluid supply system and a valve;

FIG. 6 depicts another illustrative endoscope system having a fluid supply system and a valve;

FIG. 6A depicts another illustrative endoscope system having a fluid supply system and a valve;

FIG. 7 depicts a schematic cross-section of an illustrative valve in a first position;

FIG. 8 depicts a schematic cross-section of the illustrative valve in a second position;

FIG. 9 depicts a schematic cross-section of an illustrative valve in a first position; and

FIG. 10 depicts a schematic cross-section of an illustrative valve in a second position.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal. ” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or”unless the content clearly dictates otherwise.

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. Some systems may use two separate water bottles for irrigation and lens wash while other systems may use a single water bottle for both irrigation and lens wash. As clinicians work through each case, some volume of water is depleted from the water bottle and bottles may need to be replaced one or more times over the course of the day. The process of exchanging bottles may require the user to bend or stoop down, remove the cap and associated inlet tubes from the empty bottle, and place them into a full bottle of sterile water without touching/contaminating the tubes against the external bottle or other non-sterile surfaces (e.g., so as not to create an infection risk to the patient). This may be especially difficult in the single bottle devices where multiple inlet hoses dangle from the cap when removing the cap to replace the sterile water bottle.

Additionally, having the sterile water bottles stowed on lower shelves of the carts, alongside the peristaltic pump, and other equipment may make these difficult to visualize and often, the clinician may not realize that the bottle is nearing empty until they are no longer able to deliver irrigation or lens wash to through the distal end of the scope. There is also an inherent risk associated with stowing the water bottles adjacent to the endoscope control boxes. For example, if the water bottle fails in some way (e.g., leak, burst, rupture, etc.) there may be a high risk of water running or spraying onto these high-cost control systems resulting in significant damage or destruction.

To use an individual intravenous solution bag as the primary fluid source for endoscopy (e.g., for both irrigation and lens washing), there must be a non-pressurized portion and a pressurized portion of a device. However, fluid flow must be available when requested by the user to both the pressurized portion and the non-pressurized portion continually. To accomplish this goal, previous approaches employ various separate and distinct pieces in conjunction. For instance, previous approaches may employ a port (e.g., a y-port), a separate spike, various separate tubing pieces, in conjunction with a separate metering valve, etc. However, such approaches may be prone to failure at interconnections between the separate components, occupy a large amount of space, be costly, and/or require a large amount of material (e.g., a large amount of plastic) to form each of the separate individual components.

Disclosed herein are containers, valves and tube sets that are easily viewed by the clinician, reduce the risk of contamination, and incorporate the aforementioned functions into an individual valve that is configured to allow continuous fluid flow to the non-pressurized portion of a device (e.g., an irrigation tube of an endoscope), and yet permits fluid flow for both irrigation and lens washing at the same time when desired.

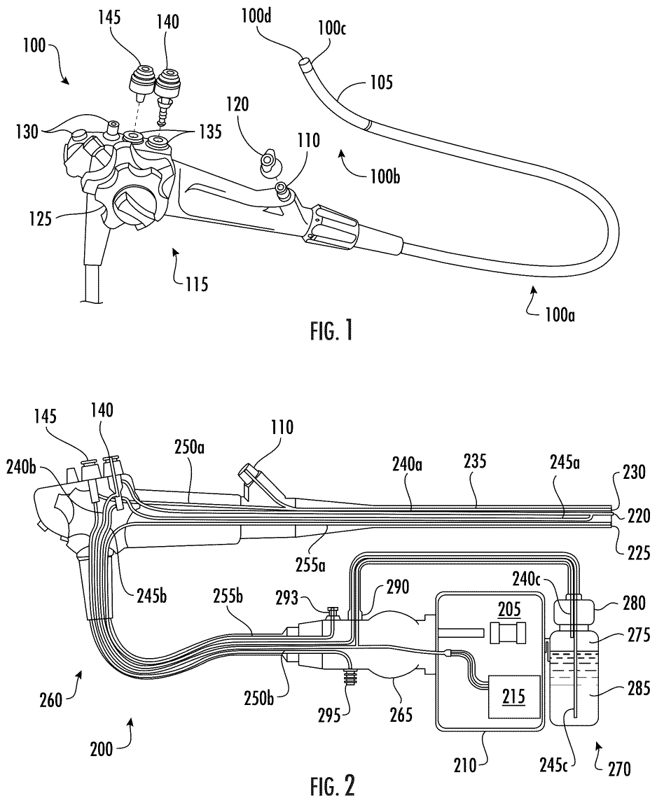

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

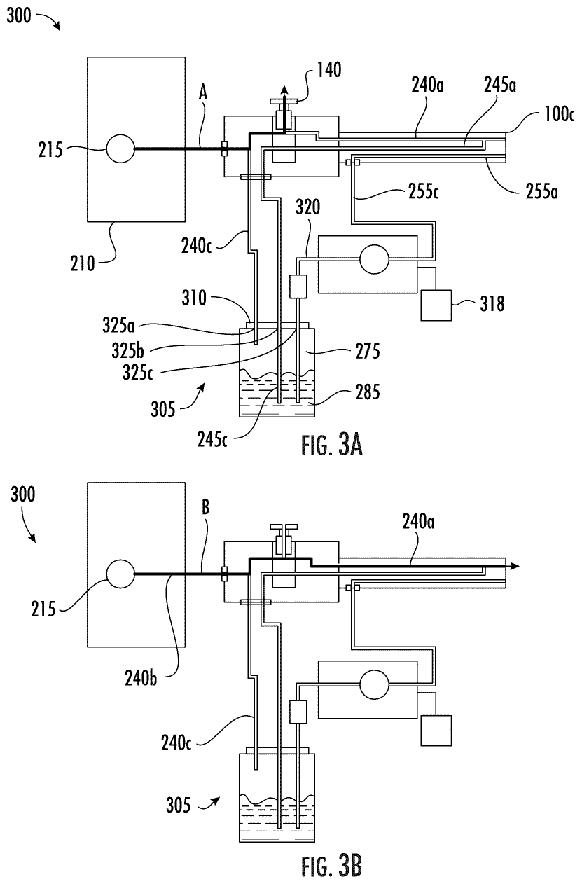

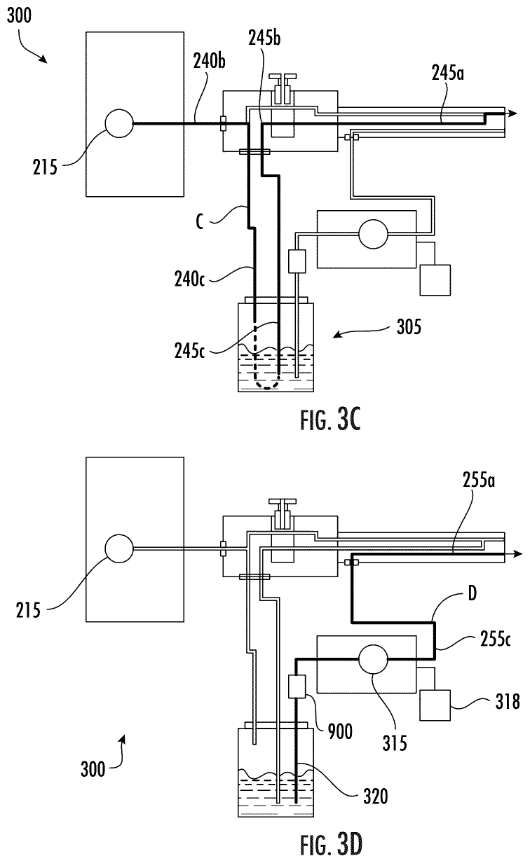

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream supply tubing for irrigation 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. Some illustrative coaxial arrangements are described in commonly assigned U.S. Patent Application Number Ser. No. 17/558,239, titled INTEGRATED CONTAINER AND TUBE SET FOR FLUID DELIVERY WITH AN ENDOSCOPE and U.S. Patent Application Number Ser. No. 17/558,256, titled TUBING ASSEMBLIES AND METHODS FOR FLUID DELIVERY, the disclosures of which are hereby incorporated by reference. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 245c, upstream irrigation supply tubing 320, and/or downstream irrigation supply tubing 255c to help prevent backflow of water from either or both of the lens wash and supply tubing for irrigation in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 245c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash supply tubing 245c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 245c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 245c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to deliver water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

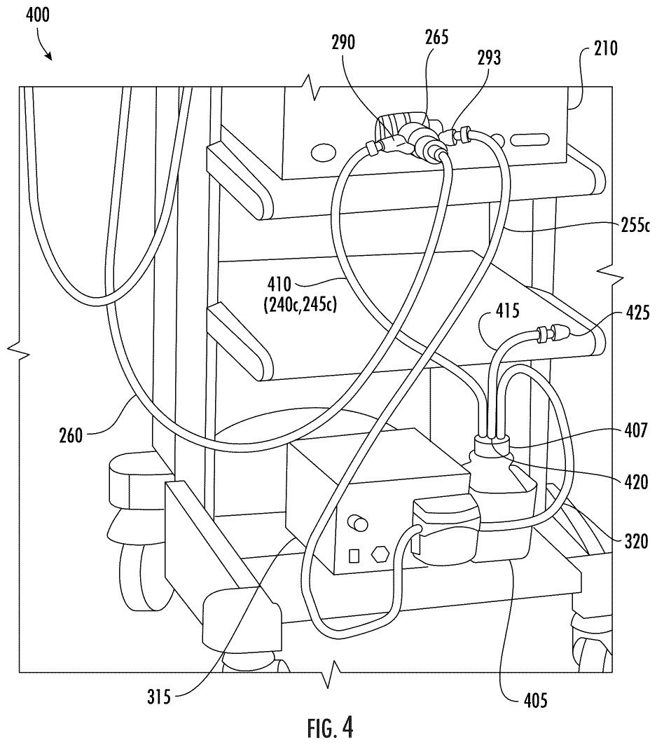

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, respectively, and alternative gas (e.g., CO₂) supply tubing 415. A length of the alternative gas supply tubing 415 passes from one end positioned in the gas gap 275 (see FIG. 2) between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. Generally, the flow of CO₂ through the endoscope 100 is similar to the flow of air. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100 and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivering gas through alternative gas (e.g., CO₂) supply tubing 415. The irrigation function may be accomplished in a similar manner as the operation described above with respect to FIG. 3D. As described above, it may be desirable to reduce opportunities for contamination to the tube set 240c, 245c, 320, 410, 415 during replacement of the water reservoir(s).

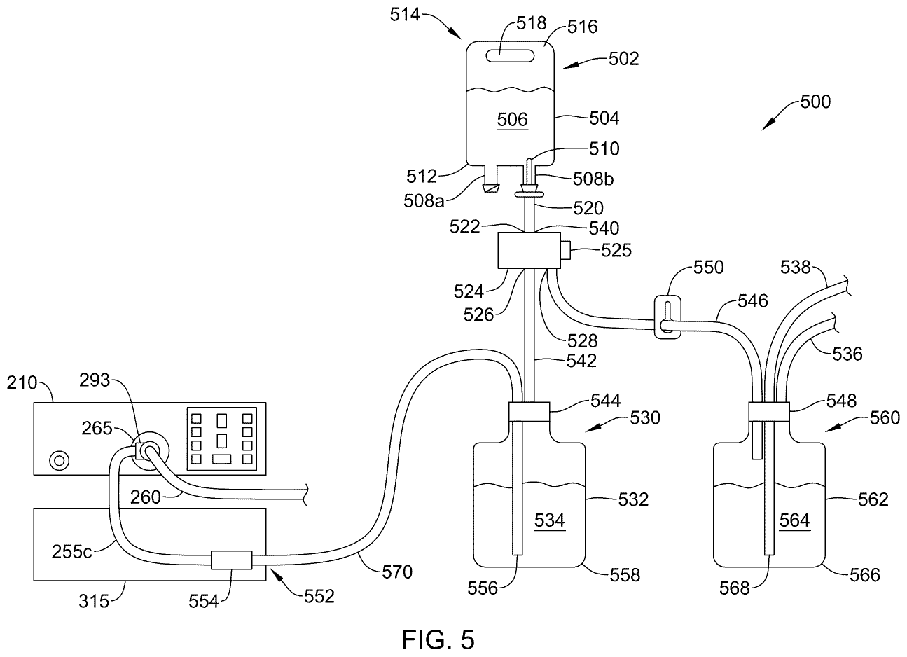

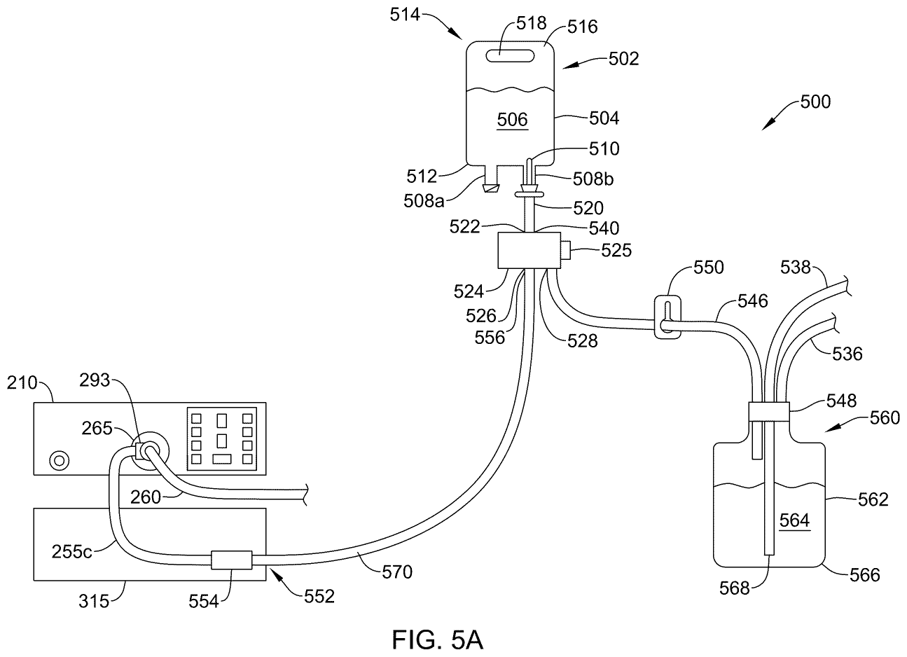

FIG. 5 depicts a schematic view of an illustrative endoscopic system 500 which may reduce the number of water reservoir changes, reduce a quantity of components as compared to previous tube sets e.g., those described above, and/or may reduce opportunities for contamination during replacement of the water reservoir(s). The system 500 may include a number of advantages over the current bottle system described above. The system 500 may include components similar to the endoscope and endoscope systems described with regard to FIGS. 1-4; however, not all features may be described or shown here.

Generally, the system 500 may include a first reservoir 502, a second reservoir 530, and a third reservoir 560. The first reservoir 502 may be configured to supply fluid for both irrigation (e.g., via the second reservoir 530) and lens wash (e.g., via the third reservoir 560). This may allow a single fluid source to be used to provide fluid for both irrigation and lens wash. While not explicitly shown, the first reservoir 502 may include printed lines, numbers, or other visual indicia to allow a user to easily determine how much fluid is left in the first reservoir 502.

The first reservoir 502 may include a first container 504 configured to hold a first volume of fluid 506. In the illustrated embodiment, the first container 504 is fluidly coupled to the second fluid reservoir 530 and may be selectively fluidly coupled to the third reservoir 560. The second reservoir 530 may include a second container 532 configured to hold a second volume of fluid 534 and the third reservoir 560 may include a third container 562 configured to hold a third volume of fluid 564.

In the illustrated embodiment, the second container 532 is fluidly coupled to the irrigation supply tubing 570 and is configured to provide fluid for irrigation. Generally, the irrigation supply tubing 570 may be a water or fluid supply line or tube for supplying water or other fluid to an endoscope. The third container 562 may be fluidly coupled to the gas and lens wash supply tubing 536, 538 and is configured to provide fluid for lens wash to the endoscope. Generally, the lens supply tubing 538 may be a water or fluid supply line or tube for supplying water or other fluid to an endoscope. In the illustrated embodiment, the gas and lens wash supply tubing 536, 538 may be arranged in a side-by-side arrangement. However, in other embodiments, the gas and lens wash supply tubing 536, 538 may be coaxially arranged. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the third reservoir 560. The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265.

The first container 504 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first container 504 may be entirely translucent, entirely opaque, or combinations thereof. In some cases, the first container 504 may be a flexible bag analogous to those utilized to deliver intravenous replacement fluid in clinical settings (for example, an intravenous (IV) fluid bag). Such bags may be readily available and familiar to the clinician as they are widely used in various sizes. The second and third containers 532, 562 may be formed from a rigid bottle. However, it is contemplated that any of the first, second, or third containers 504, 532, 562 may be formed from a flexible bag or a rigid bottle, as desired. Additionally, while the illustrated embodiment in FIG. 5 employs a second reservoir 530 with the second container 532 that is configured to hold the second volume of fluid 534, in some embodiments the second reservoir 530 may be omitted and the first container 504 may be fluidically coupled (e.g., via the irrigation supply tubing 570) to a downstream component such as the irrigation pump 315. For instance, as illustrated in FIG. 5A, the second reservoir 530 is omitted so that the first end 556 of the upstream irrigation supply tubing 570 directly connects end 556 of the tube 570 directly connects with the second port 526 of the valve body 527. That is, FIG. 5A is analogous to FIG. 5, but with the change that the second reservoir 530 is omitted so that the first end 556 of the upstream irrigation supply tubing 570 directly connects end 556 of the tube 570 directly connects with the second port 526 of the valve body 527. It is contemplated that this may further reduce connection points in the fluid circuit.

The volume of the first, second, and/or third container 504, 532, 562 may be variable. For example, the volume of the first, second, and/or third container 504, 532, 562 may be 500 milliliters (mL) or greater, 1000 mL or greater, 2000 mL or greater, 3000 mL, 4000 mL or greater, etc. The volume may be less than 500 mL or greater than 4000 mL, as desired. One, two, or all of the first, second, and/or third containers 504, 532, 562 may be pre-filled (e.g., prior to entering the procedure suite or at the time of manufacturing) with water or other fluid. In some cases, the clinician may select the reservoir(s) 502, 530, 560 from a plurality of differently sized available reservoirs based on the number and/or types of procedures expected for a typical or the specific day. In the illustrated embodiments, the first reservoir 502 may supply fluid to the second reservoir 530 and the third reservoir 560. By selecting a first reservoir 502 having a volume large enough to accommodate an entire day of procedures, the need for replacing the sterile fluid source (e.g., the first reservoir 502) may be reduced or eliminated. In some cases, the first reservoir 502 may be used to periodically refill the second reservoir 530 and/or third reservoir 560. Thus, the volume of the first reservoir 502 may be greater than the volume of the second reservoir 530 and/or third reservoir 560, although this is not required.

It is contemplated that flexible bags may utilize less plastic (or other material) than a bottle designed to hold a similar amount of fluid. Thus, the use of a flexible bag as a fluid reservoir 502, 530, 560 may increase the level of environmental sustainability of the system 500. For example, if the user sets up the system with a 3000 mL (3 liter) bag reservoir 502 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 502 may further include one or more ports 508a, 508b, such as, but not limited to a spike port or a septum port, extending from and in selective fluid communication with an interior of the first container 504. The ports 508a, 508b may be formed as a monolithic structure with the first container 504. The ports 508a, 508b may be generally tubular structures with each port 508a, 508b defining a lumen extending therethrough. The lumens of the ports 508a, 508b may be configured to selectively fluidly couple the interior of the first container 504 with another component, such as, but not limited to, a fluid or water supply tube 520 and/or a valve 524, as described herein. In some embodiments, the ports 508a, 508b may be positioned adjacent to a bottom end 512 of the first reservoir 502. However, this is not required. The ports 508a, 508b may be positioned in other locations, as desired. If the ports 508a, 508b are positioned at a location other than the bottom end 512 of the first container 504, a dip tube or tube extension may be required to access the fluid at the bottom of the first container 504. In some cases, at least one port 508b may be configured to be coupled to the water supply tube 520 while another port 508a may be configured to allow the user to add additives to the fluid 506. While the first reservoir 502 is illustrated as including two ports 508a, 508b, the first reservoir 502 may include one port or more than two ports, as desired.

While not explicitly shown, the ports 508a, 508b may each include a removable cap or seal configured to form a fluid tight seal with the port 508a, 508b. The removable cap or seal may help to maintain the sterility of the ports 508a, 508b. The removable cap or seal may be coupled to a free end of the ports 508a, 508b using a number of different techniques. For example, the cap or seal may be coupled to the port 508a, 508b using a threaded engagement, a friction fit, a snap fit, etc. In other instances, the cap or seal may be removed through a twisting motion configured to break the cap or seal from the port 508a, 508b. Once the cap or seal has been removed, the port 508a, 508b may be pierced with a spike tip or spike port adaptor 510 that is coupled to the water supply tube 520 and/or the valve 524. For example, in addition to the removable cap or seal, the port 508a, 508b may include an internal seal disposed within a lumen of the port 508a, 508b that may be punctured or pierced by the spike port adaptor 510. The internal seal may be configured to prevent fluid 506 from leaking from the first container 504 prior to the spike port adaptor 510 being inserted into the port 508a, 508b. In some embodiments, the internal seal may be self-sealing such that upon removal of the spike port adaptor 510 fluid is prevented from leaking from the port 508a, 508b. The outer surface of the spike port adaptor 510 may form an interference fit with the inner surface of the port 508a, 508b. It is contemplated that the spike port adaptor 510 may be inserted into one of the ports 508a, 508b utilizing universally used aseptic techniques such as those used with IV fluid bags and/or it is contemplated that the spike port adaptor 510 may be integrated in the valve 524. This may help reduce infection risk by maintaining sterile components, not introducing contaminants into the fluid 506, etc. It is further contemplated that additives may be added to the fluid 506 using similar aseptic techniques via one of the ports 508a, 508b.

The first reservoir 502 may include a handle 516 positioned adjacent to a top portion 514 thereof. The handle 516 may define an opening or through hole 518 for receiving a hand or hook therethrough to carry the first reservoir 502. In some cases, the handle 516 may include an undulating surface configured to provide a more ergonomic grip for the user. It is contemplated that the handle 516 may be formed from a similar material as the first container 504 or a different material, as desired. In some examples, the handle 516 may be formed from polyethylene terephthalate (PET), polypropylene (PP), plasticized polyvinyl chloride (PVC), etc. The handle 516 may allow the first reservoir 502 to be hung from a hook, such as, but not limited to an IV stand. Hanging the first reservoir 502 may allow the first reservoir 502 to be positioned above the level of an endoscope cart which may enable the user to see the fluid 506 level at any time. This may help the clinician avoid running out of fluid during a procedure. Additionally, elevating the reservoir may eliminate the need for the clinician to bend or stoop during setup of the system 500 and/or to change the first reservoir 502. In some cases, head pressure generated from the elevating the first reservoir 502 may enable rapid priming of the irrigation circuit (and/or lens wash circuit if so connected) which may save time during setup. It is further contemplated that hanging the first reservoir 502 from a hook or IV stand may allow the first reservoir 502 to be positioned away from expensive capital equipment thus reducing or eliminating the potential for fluid running or flowing inadvertently onto the capital equipment and causing damage or destruction.

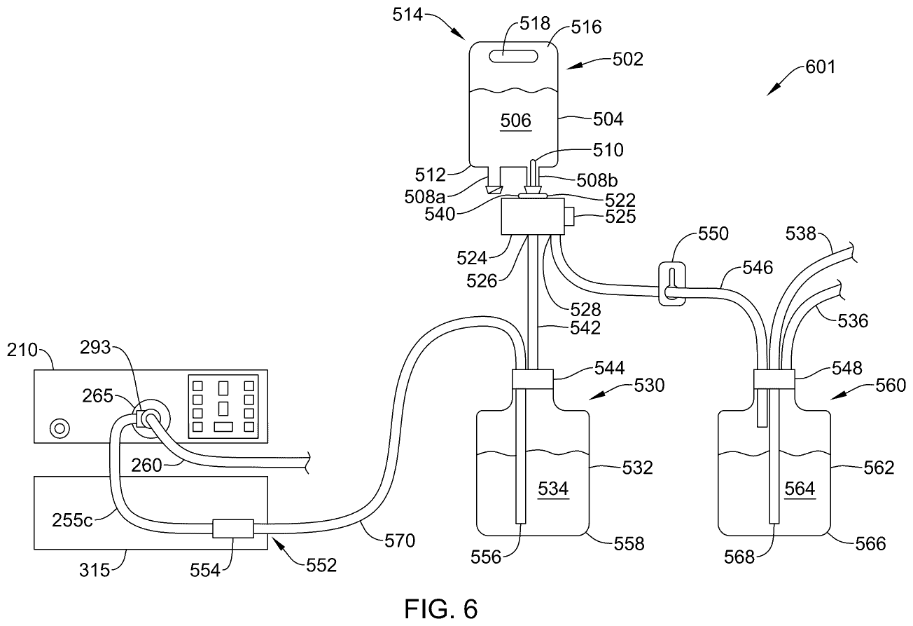

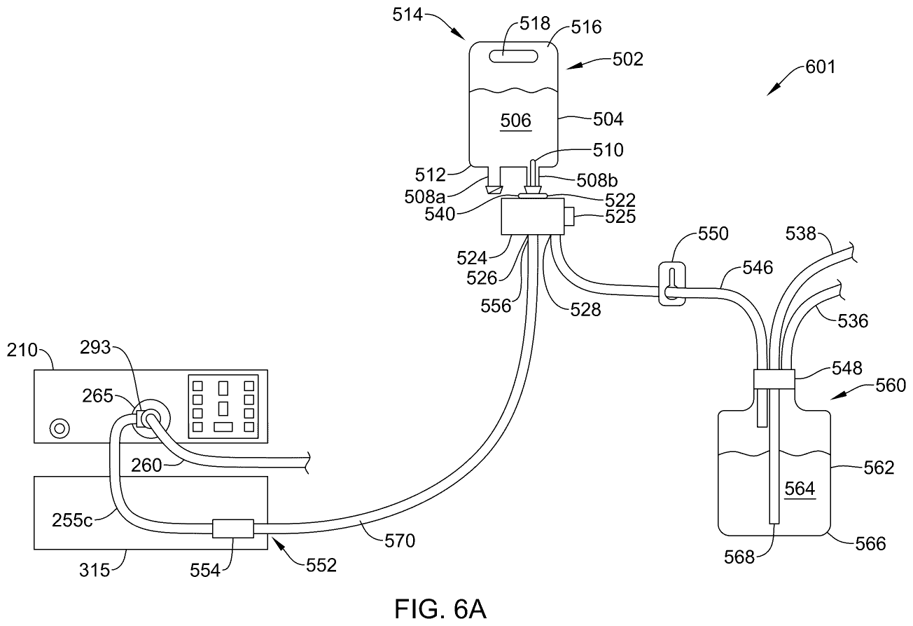

As illustrated in FIG. 5, in some embodiments the first reservoir 502 may be connected in fluid communication with a lumen of the water supply tube 520. The water supply tube 520 may extend from a first end that is coupled to the spike port adaptor 510 to a second end 522 which is coupled to a valve 524. However, in some embodiments the first reservoir 502 may be connected in direct fluidic communication with the valve 524. Stated differently, in some embodiments the spike port adaptor 510 can be included in the valve 524, for instance as described with respect to FIG. 6. It is contemplated that the valve 524 may be a separate structure coupled to the container first reservoir 502 or may be formed as a unitary structure with the first reservoir 502.

In some embodiments, the valve 524 may include a branched connector such as a “Y” connector or a “T” connector having a first port 540 (e.g., a first inlet or a first inlet leg) defining a first fluid inlet, a second port 526 (e.g., a first outlet or first outlet leg) defining a first fluid outlet, and a third port 528 (e.g., a second outlet or second outlet leg) defining a second fluid outlet. However, it is contemplated that the valve 524 may include more than one fluid inlet and more than two fluid outlets, if so desired.

The first port 540 may be configured to receive fluid from a container such as the first container 504. For instance, the first port 540 of the valve 524 may be coupled to the second end 522 of the water supply tube 520 to receive a flow of fluid from the first reservoir 502. The valve 524 may be configured to divert some of the flow of fluid to the second port 526 and some of the flow of fluid to the third port 528. The second port 526 may be fluidly coupled with a lumen of a second fluid supply tube 542. The second fluid supply tube 542 extends from a second end coupled with a cap 544 of the second container 532 to a first end coupled with the second port 526. The second water supply tube 542 may be configured to selectively fluidly couple the first reservoir 502 with the second reservoir 530. For example, the second water supply tube 542 may extend through an opening or port in the cap 544 to allow fluid to pass from the lumen of the second water supply tube 542 into the interior of the second container 532. The opening in the cap 544 may include a seal or O-ring configured to seal the cap 544 about the tubing 542 in a fluid and pressure tight manner. In some cases, the second water supply tube 542 may include a flow control mechanism (not explicitly shown), such as, but not limited to a valve, a one-way valve, a clamp, a stopcock, etc., to selectively allow a flow of fluid from the first reservoir 502 to the second reservoir 530.

The third port 528 may be fluidly coupled with a lumen of a third fluid supply tube 546. The third fluid supply tube 546 extends from a second end coupled with a cap 548 of the third container 562 to a first end coupled with the third port 528. The third fluid supply tube 546 may be configured to selectively fluidly couple the first reservoir 502 with the third reservoir 560. For example, the third fluid supply tube 546 may extend through an opening or port in the cap 548 to allow fluid to pass from the lumen of the third fluid supply tube 546 into the interior of the third container 562. The opening in the cap 548 may include a seal or O-ring configured to seal the cap 548 about the tubing 546 in a fluid and pressure tight manner. In some cases, the third fluid supply tube 546 may include a flow control mechanism 550, such as, but not limited to a valve, a one-way valve, a clamp, a stopcock, etc., to selectively allow a flow of fluid from the first reservoir 502 to the third reservoir 560. It is further contemplated that the flow control mechanism 550 may prevent a flow of air/gas from the third reservoir 560 to the first and/or second reservoirs 502, 530. It is contemplated that the flow control mechanism 550 may be opened only when it is desired to add fluid to the third container 562 from the first container 504. However, in some cases the flow control mechanism 550 can be omitted from the third fluid supply tube 546. For instance, at least due to the presence of the valve 524, some embodiments herein can utilize the third fluid supply tube 546 that is without a flow control mechanism other than the valve 524. It is contemplated that use of the valve 524 as a flow control mechanism for the third fluid supply tube 546 may reduce connection points in the fluid circuit. Fluid may be added to the second container 532 while the irrigation pump 315 is running or while the irrigation pump 315 is idle, as desired.

The valve 524 may be positioned such that the first port 540 is upstream of the second and third ports 526, 528 relative to a flow of fluid from the first reservoir 502. In some embodiments, the valve 524 and the spike port adaptor 510 may be molded or formed as a single monolithic structure. For instance, the first port 540 may include the spike port adaptor 510 that is configured to be inserted into a port such as the port 508b of the first container 504, as illustrated in FIG. 6. It is contemplated that this may reduce connection points in the fluid circuit.

The upstream irrigation supply tubing 570 extends from a second end region 552 external to the second container 532 and positioned within a pump head 554 of the peristaltic irrigation pump 315 to a first end 556. The first end 556 of the upstream irrigation supply tubing 570 extends through an opening in the cap 544 of the second reservoir 530 and is positioned adjacent to a bottom portion 558 of the second container 532. The opening in the cap 544 may include a seal or O-ring configured to seal the cap 544 about the tubing 570 in a fluid and pressure tight manner. The second end of the upstream irrigation supply tubing 570 is configured to be fluidly coupled with an irrigation lumen of the endoscope 100. When irrigation water is required, fluid is pumped from the second container 532 by operating the irrigation pump 315, such as by depressing a footswitch (not shown), and flows from the second reservoir 530, through the upstream irrigation supply tubing 570, through the downstream irrigation supply tubing 255c, through the irrigation connection 293, through the irrigation feed line 255b in the umbilical 260, and down the irrigation supply line 255a in the shaft 100a of the endoscope to the distal tip 100c. Fluid 506 from the first reservoir 502 may be continuously supplied via the valve 524 to the second reservoir 530 as fluid 534 is pumped from the second reservoir 530. Alternatively, a flow control mechanism positioned in line with the second fluid supply tube 542 and the valve 524 may be opened to refill the second reservoir 530 on demand.

The downstream irrigation supply tubing 255c may include a loaded check valve or flow control valve (not explicitly shown) positioned in line with the downstream irrigation supply tubing 255c. The flow control valve may prevent the unintentional flow of fluid from the first container 504 to the endoscope 100. In some cases, the flow control valve may be configured to open when the pressure within the downstream irrigation supply line 255c reaches a predetermined minimum pressure. The flow control valve may also prevent fluid from leaking from the downstream irrigation supply tubing 255c when the endoscope 100 is changed between patients.

The gas supply tubing 536 extends from a second end external to the third container 562 and through an opening in the cap 548 thereof. The gas supply tubing 536 may extend into the interior of the third container 562 and terminate within a reservoir gap (e.g., above the level of the fluid 564). However, in some cases, the gas supply tubing 536 may terminate within the fluid 564. A lumen extends through the gas supply tubing 536 for receiving a flow of air and/or gas therethrough. The lumen of the gas supply tubing 536 may be in operative fluid communication with a top portion of the interior of the third container 562. The lens wash supply tubing 538 extends from a second end external to the third reservoir 560 to a first end 568 in fluid communication with a bottom portion 566 of the third container 562. A lumen extends through the lens wash supply tubing 538 for receiving a flow of fluid therethrough. In the illustrated embodiment, the gas supply tubing 536 and the water supply tubing 538 may couple to the third container 562 through separate openings in the cap 548. However, this is not required. In some cases, the gas supply tubing 536 and the lens wash supply tubing 538 may be coaxially arranged. The openings in the cap 548 may include a seal or O-ring configured to seal the cap 548 about the tubing 536, 538 in a fluid and pressure tight manner.

While not explicitly shown, in some embodiments, an alternative gas supply tube may be coupled to an alternative gas supply (e.g., CO₂ hospital house gas source) and fluidly coupled to the third reservoir 560. The alternative gas supply may be used to pressurize the third container 562 to supply lens wash to the endoscope 100 and/or to provide insufflation.

Fluid 506 from the first reservoir 502 may be supplied to the third reservoir 560 by variation of a position of the valve 524. For instance, fluid 506 from the first reservoir 502 may be supplied to the third reservoir 560 when the plunger is in a second position at which the first flow path and the second flow path are open. Additionally, in some instances, fluid 506 from the first reservoir 502 may be supplied to the third reservoir 560 by opening or releasing the flow control mechanism 550 positioned in line with the third fluid supply tube 546. In some cases, the pressure within the third reservoir 560 may need to be relieved prior to allowing fluid to flow from the first reservoir 502 to the third reservoir 560. It is contemplated that a pressure relief valve or three-way stopcock may be provided in the gas supply tubing 536 to allow the pressure in the third reservoir 560 to be relieved. As the pressurized third container 562 is fluidly isolated from the first container 504 when the plunger is in a given position (e.g., in a first position) and/or when the flow control mechanism 550 is closed, it is contemplated that the clinician may replace the first reservoir 502 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

In some embodiments, the irrigation pump 315 may be omitted. For example, the first reservoir 502 may be inserted into a compression sleeve. When irrigation fluid is desired, the compression sleeve may be activated to exert pressure on an outer surface of the first reservoir 502 and to provide the required pressure to perform irrigation at the distal end of the endoscope 100.

In some embodiments, a lens wash supply tubing may be coupled to the first port 508a. In this embodiment, the lens wash supply tubing and the irrigation supply tubing may be coupled to the same reservoir 502. The first reservoir 502 may be inserted into a compression sleeve, as described above. The pressure exerted by the compression sleeve may be sufficient to supply pressurized fluid for both irrigation and lens wash to the endoscope without the use of a secondary pump (such as an irrigation pump) or pressure source (such as an insufflator).

If there is a need to replace the first reservoir 502 with a new full bag, for example when the first reservoir 502 is empty or near empty, the user may hang the new bag near the first reservoir 502 to be replaced. The user may then disengage the spike port adaptor 510 from the port 508b and insert the spike port adaptor 510 into a port of the new bag. This may be performed without requiring the clinician to bend or stoop to access the first reservoir 502. The port 508b may self-seal to prevent fluid leaks from the first reservoir 502 being replaced. This method of replacing the first reservoir 502 may have a lower risk of introducing contaminants into the systems relative to traditional bottle systems. For example, the change out method described herein may allow the first reservoir 502 to be changed out without having tubing tangling from a cap (as in a bottle system). Further, the system 500 may remain largely closed as the first reservoir 502 is changed out.

In some embodiments, it may be desirable to supply irrigation fluid without a dedicated pump or through pressurization of the fluid reservoir.

FIG. 6 depicts another illustrative endoscope system 501 having a fluid supply system and a valve. The system 600 may include components similar to the endoscope and endoscope systems described with regard to FIGS. 1-4; however, not all features may be described or shown here. FIG. 6 is analogous to FIG. 5, but with the change that the valve 524 and the spike port adaptor 510 are molded or formed as a single monolithic structure that is configured to be directly inserted into a port such as the port 508b of the first container 504, as illustrated in FIG. 6. That is, the valve 524 may be configured (e.g., with a spike port adaptor 510 extending from the first port 540 a distance outside of the valve body 527) to be directly (e.g., in the absence of an intervening component such as the water supply tube 520) inserted in a port of the container, as illustrated in FIG. 6. It is contemplated that this may further reduce connection points in the fluid circuit. While the illustrated embodiment in FIG. 6 employs a second reservoir 530 with the second container 532 that is configured to hold the second volume of fluid 534, in some embodiments the second reservoir 530 may be omitted and the first container 504 may be fluidically coupled (e.g., via the irrigation supply tubing 570) to a downstream component such as the irrigation pump 315. For instance, as illustrated in FIG. 6A, the second reservoir 530 is omitted so that the first end 556 of the upstream irrigation supply tubing 570 directly connects end 556 of the tube 570 directly connects with the second port 526 of the valve body 527. That is, FIG. 6A is analogous to FIG. 6, but with the change that the second reservoir 530 is omitted so that the first end 556 of the upstream irrigation supply tubing 570 directly connects end 556 of the tube 570 directly connects with the second port 526 of the valve body 527. It is contemplated that this may further reduce connection points in the fluid circuit.

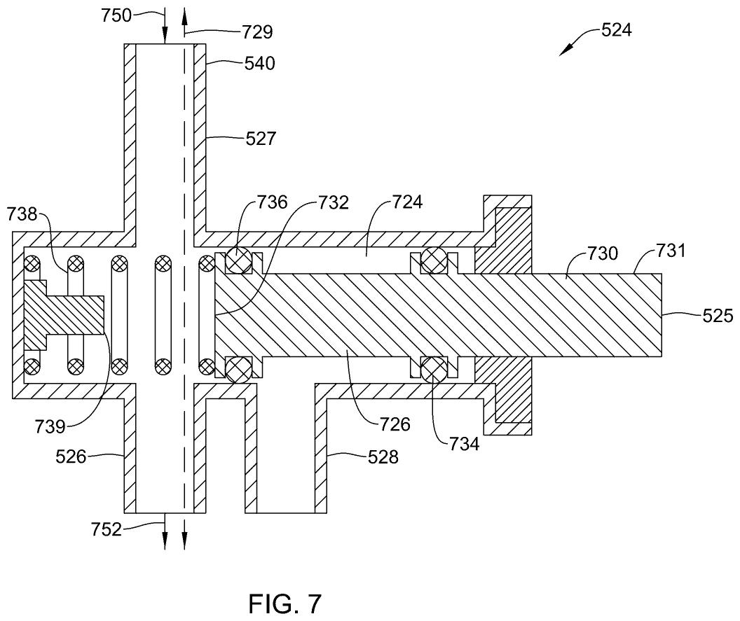

FIG. 7 depicts a schematic cross-section of an illustrative valve 524 in a first position. As illustrated in FIG. 7, the valve 524 includes a valve body 527. The valve body 527 may be a rigid component that is formed of plastic, metal, and/or another type of material. The valve body 527 may define various ports and a passage 724 extending between the various ports. For instance, the valve body 527 can define a first port 540, a second port 526, a third port 528, and the passage 724 extending between the first port 540, the second port 526, and the third port 528. A retention cap 733 can retain a plunger 730 within the valve body 527. In some embodiments, the retention cap 733 can include a snap-lock (not illustrated) or other type of mechanism to retain the plunger 730 within the valve body 527. The retention cap 733 can glued, threaded, clipped into or otherwise affixed to the valve body 527.

As described herein, the first port 540 (e.g., an inlet port) may be configured to receive fluid (e.g., represented as arrow 750) from a container such as the first container 504. The second port 526 may be in fluid communication with the first port 540 via a first flow path. The first flow path may extend along a portion of the passage 724 between the first port 540 and the second port 526. As illustrated in FIG. 7, the first flow path between the first port 540 and the second port 526 is open (e.g., permits fluid flow along the first flow path) when a plunger 730 is in the first position. As such, at least a portion of the fluid 750 received at the first port 540 can be communicated via the first flow path to the second port 526 at which point the fluid (represented as arrow 752) can exit the valve 524 via the second port 526. For instance, the fluid 752 can be provided to lumen of an irrigation supply tubing such as the lumen of the irrigation supply tubing 570 and/or the lumen of a second fluid supply tube 542, as described with respect to FIG. 5 and FIG. 6. In some cases, the fluid 752 can be provided continuously to lumen of an irrigation supply tubing such as the lumen of the irrigation supply tubing 570 and/or the lumen of a second fluid supply tube 542 to promote as aspects herein, such as continually supplying fluid via the first flow path to the endoscope.

The third port 528 may be configured to be in fluid communication with the first port 540 via a second flow path. The second flow path may extend along a portion of the passage 724 between the first port 540 and the third port 528. As illustrated in FIG. 7, the second flow path between the first port 540 and the third port 528 is closed (e.g., does not permit fluid to flow along the flow path) when the plunger 730 is in the first position. That is, as illustrated in FIG. 6, when the valve 524 is a first position, the first flow path (e.g., an irrigation flow path) may be open and the second flow path (e.g., a lens wash flow path) may be closed. As such, in the illustrated embodiment of FIG. 6, the fluid 752 may exit the second port 526 via the first flow path when the valve 524 is in the first position; however, no fluid exits the third port 528 when the plunger 730 of the valve 524 is in the first position.

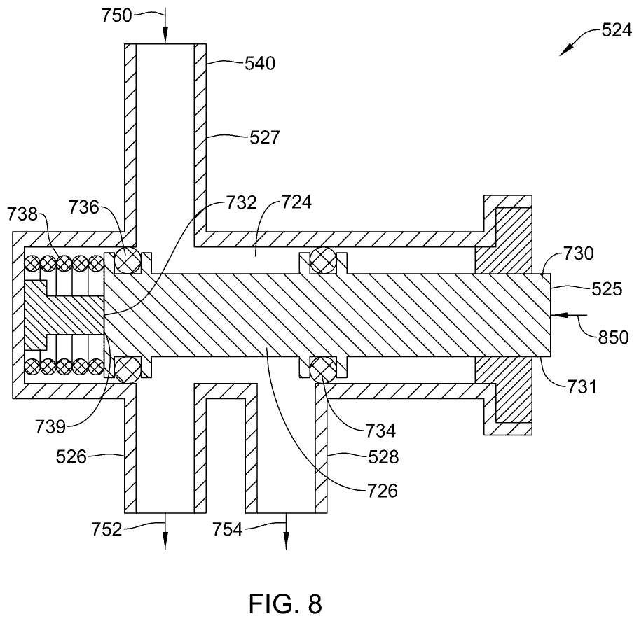

For instance, the valve 524 may include a plunger 730 that is disposed in a portion of the passage 724. The plunger 730 may be a formed of a monolithic member or various components. The plunger 730 may be configured to selectively move (e.g., move axially along the passage 724) to open or close a flow path (e.g., to selectively open or close the second flow path). For instance, the plunger 730 may piston or double-piston shaped member that is configured with a plurality of seals to permit the plunger 730 to selectively open or close a flow path depending on a position of the plunger 730. In some embodiments, the plunger 730 may be a double-piston shaped plunger having a first seal 734 at or relatively proximate to a first end 731 of the dual piston and a second seal 736 at or relatively proximate to a second end 732 of the dual piston that is opposite the first end 731. The first seal 734 may be configured to prevent fluid from flowing out of the valve body 527 near the first end 731 of the plunger 730 when the plunger is in the first position and when the plunger is in the second position. In some embodiments, a second seal 736 may be located along an interface between a second end 732 (e.g., a bottom portion) of the plunger 730 and the valve body 527 that is adjacent to the second end 732 of the plunger 730, as illustrated in FIG. 6. The second seal 736 may be configured to prevent pressurized gas (e.g., air and/or CO₂) from backflowing along the second fluid path into the container through the first port 540 when the plunger 730 is in the first position. The seals 734, 736, may include a gasket, an O-ring, a compressible elastomeric member, etc. For instance, in some embodiments, the first seal and the second seal may each comprise a respective a wiper seal or a respective beaded seal, among other possibilities. In some embodiments, the seal may be overmolded seals that are formed on or in conjunction with formation of the plunger 730, thereby forming a unitary plunger including the seals (e.g., overmolded seals). In some embodiments, the plunger 730 may be configured as a double-piston plunger having a center portion 726 with a diameter (e.g., extending in the same direction as the axis 729) that is less than a first diameter at a first seal 743 and is less than a second diameter at a second seal of the double-piston plunger, as illustrated in FIG. 7. The center portion 726 may extend between the first seal 734 and the second seal 736. Thus, the first seal 734 may be spaced by a distance away from the second seal 736, as illustrated in FIG. 7. As such, the double-piston plunger may selectively permit fluid flow along both a first flow path and a second flow path that extend past or though the center portion 726 of the double-piston plunger when the plunger 730 is in a second position, as illustrated in FIG. 8.

The valve 524 can include a biasing mechanism 738. The biasing mechanism 738, such as, but not limited to, a spring, may be positioned along the passage 724 to bias the plunger 730 to the first position, as illustrated in FIG. 6. For instance, the biasing mechanism can be positioned about a biasing retention member 739 between a second end 732 (e.g., a bottom (innermost) portion) of the plunger 730 and the bottom (innermost) portion of the passage 724.

The plunger 730 can include a first end 731 that extends a distance outside of the valve body 527 and forms a lens wash button 525. In some embodiments, the plunger 730 may be configured to move from the first position (e.g., as illustrated in FIG. 6) to a second position (e.g., as illustrated in FIG. 7) responsive to actuation (e.g., as represented by element 850) of the lens wash button 525. When the plunger 730 is in the second position, both the first flow path and the second flow path may be open. As such, both an irrigation circuit and an lens washing circuit can be provided fluid at the same time. For instance, FIG. 8 depicts a schematic cross-section of the illustrative valve 524 in a second position. As illustrated in FIG. 8, the fluid 752 (e.g., fluid for the irrigation circuit) can exit the second port 526 and the fluid 754 (e.g., fluid for the lens wash circuit) can exit the third port 528 at the same time when the plunger 730 is in the second position. It is contemplated that providing the irrigation circuit and the lens washing circuit with fluid at the same time can enhance an irrigation and/or lens washing capability of an endoscope, for instance, as compared to other approaches that alternatively provide fluid either to an irrigation circuit or to a lens washing circuit.

In some embodiments, the plunger 730 may be configured to move from the second position to the first position responsive to release of the lens wash button 525. For instance, the biasing mechanism 738 can bias the plunger 730 to move from the second position to the first portion responsive to release of the lens wash button 525.

In some embodiments, the first port 540 may be located on a first side of the valve body 527. In some embodiments, the second port 526 and the third port 528 may be located on a second side of the valve body 527. For instance, the first port 540 may be located on a first side of the valve body 527 and each of the second port 526 and the third port 528 may be located on a second side of the valve body 527 that is opposite the first side of the valve body 527.

In some embodiments, the second port 526 or the third port 528 may be coupled to a lumen of lens wash tubing (e.g., lens wash tubing 536, 538). In some embodiments, the other of the second port 526 or the third port 528 is coupled to a lumen of irrigation supply tubing (e.g., irrigation supply tubing 570).

In some embodiments, the first port 540 and one of the second port 526 or the third port 528 may be co-located along a common axis. For instance, as illustrated in FIG. 7, the first port 540 and the second port 526 may be co-located along the common axis 729. The common axis 729 can extend along a flow path such as the first flow path. Having the first port 540 and one of the second port 526 or the third port 528 extend along the common axis 729 (e.g., extend along a flow path that is coextensive with the common axis 729) may promote readily providing the fluid 752 (e.g., irrigation fluid) via the valve 524 to an endoscope. In such embodiments, the other port (e.g., the third port 528) may be located off-axis from the common axis 729. Having the other port be located off or outside of the common axis can promote aspects herein such as promoting the selective flow of fluid (e.g., lens washing fluid) via a flow path (e.g., the second flow path) to an endoscope. As illustrated in FIGS. 7 and 8, the common-axis 729 can extend along a path that is outside of (e.g., does not extend through) the plunger 730 when the plunger is in a first position. However, other configurations such as those described herein are possible.

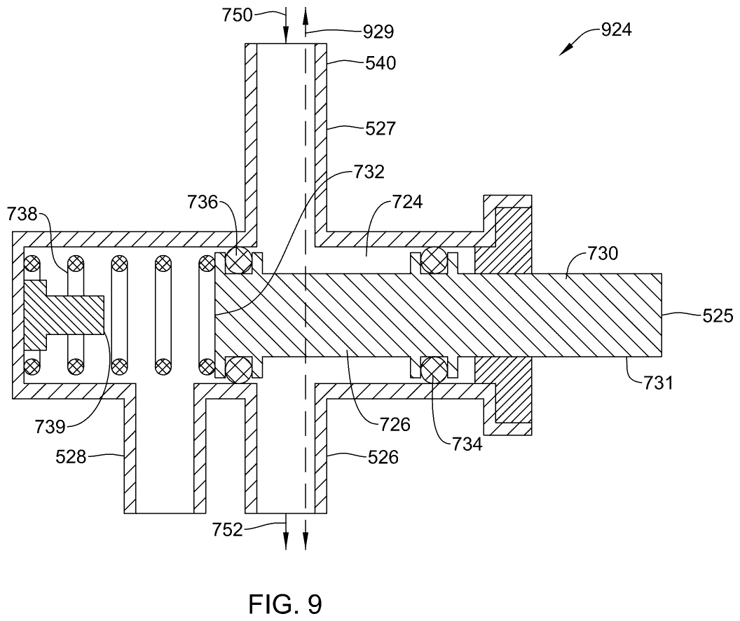

For instance, FIG. 9 depicts a schematic cross-section of an illustrative valve 924 in a first position. The valve 924 illustrated in FIG. 9 is analogous to the valve 524 illustrated in FIG. 7, but with the change that the first port 540 and the second port 526 are co-located along a common axis 929 that extends through a portion of the plunger 730 when the plunger 730 is in the first position, as illustrated in FIG. 9. In such embodiments, the other port (e.g., the third port 528) may be located off-axis from the common axis 929. Having the other port be located off or outside of the common axis can promote aspects herein such as promoting the selective flow of fluid (e.g., lens washing fluid) via a flow path (e.g., the second flow path) to an endoscope.

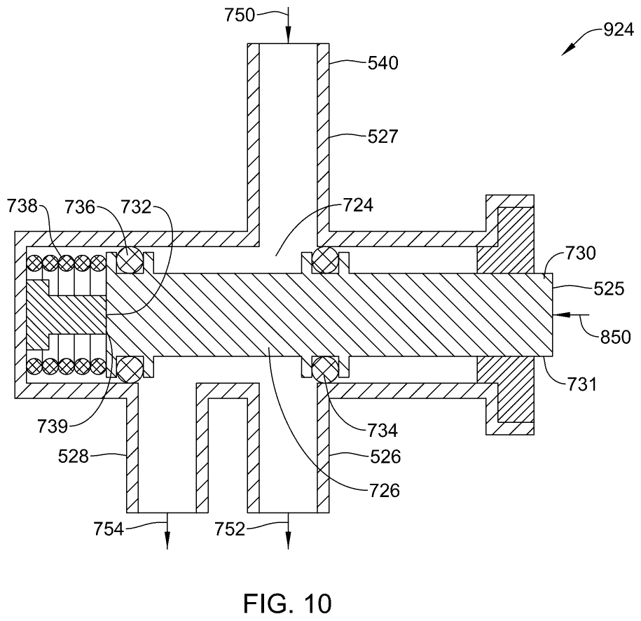

FIG. 10 depicts a schematic cross-section of an illustrative valve in a second position. The valve 924 illustrated in FIG. 10 is analogous to the valve 524 in FIG. 8, but with the change that the first port 540 and the second port 526 are co-located along a common axis 929 that extends through a portion of the plunger 730 when the plunger 730 is in the first position, as illustrated in FIG. 9. As illustrated in FIG. 10, the fluid 752 (e.g., fluid for the irrigation circuit) can exit the second port 526 and the fluid 754 (e.g., fluid for the lens wash circuit) can exit the third port 528 at the same time when the plunger 730 is in the second position. It is contemplated that providing the irrigation circuit and the lens washing circuit with fluid at the same time can enhance an irrigation and/or lens washing capability of an endoscope, for instance, as compared to other approaches that alternatively provide fluid either to an irrigation circuit or to a lens washing circuit. While the second port 526 and the third port 528 are in different locations in the valve 924 than in the valve 524, it is understood that the second port 526 and the third port 528 in FIGS. 9-10 can function in a similar manner, for instance as described with respect to FIG. 5-8. For instance, the second port 526 in FIG. 9-10 can be configured to provide the fluid 752 (e.g., irrigation fluid) to lumen of an irrigation supply tubing such as the lumen of the irrigation supply tubing 570 and/or the lumen of a second fluid supply tube 542, as described with respect to FIG. 5 and FIG. 6, when the valve 924 is in a first position and when the valve 924 is in a second position. As illustrated in FIG. 10, the fluid 752 (e.g., fluid for the irrigation circuit) can exit the second port 526 and the fluid 754 (e.g., fluid for the lens wash circuit) can exit the third port 528 at the same time when the plunger 730 is in the second position.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A valve for fluidly connecting a lumen in an endoscope to a fluid reservoir, the valve comprising:
a valve body defining:
a first port configured to receive fluid from a container;
a second port configured to be in fluid communication with the first port via a first flow path;
a third port configured to be in fluid communication with the first port via a second flow path;
a passage extending between the first port, the second port, and the third port;
a plunger configured to move in the passage between:
a first position at which the first flow path is open and the second flow path is closed; and
a second position at which the first flow path and the second flow path are open; and
a biasing mechanism configured to bias the plunger to the first position.

2. The valve of claim 1, wherein the first port includes a spike port adaptor that is configured to be inserted into a port of the container.

3. The valve of claim 1, wherein a first end of the plunger extends a distance outside of the valve body and forms a lens wash button.

4. The valve of claim 3, wherein the plunger is configured to move from the first position to the second position responsive to actuation of the lens wash button.

5. The valve of claim 4, wherein the plunger is configured to move from the second position to the first position responsive to release of the lens wash button.

6. The valve of claim 1, wherein the biasing mechanism is a spring.

7. The valve of claim 1, wherein the container is a flexible bag.

8. The valve of claim 1, wherein the plunger is configured as a double-piston plunger including a first seal and a second seal, and wherein the double-piston plunger includes a center portion with a diameter that is less than a diameter at the first seal and is less than a diameter at the second seal.

9. The valve of claim 1, wherein the first port is located on a first side of the valve body and wherein the second port and the third port are located on a second side of the valve body.

10. The valve of claim 9, wherein the first side is opposite the second side.

11. The valve of claim 1, wherein the second port or the third port is coupled to a lumen of lens wash tubing.

12. The valve of claim 11, wherein the other of the second port or the third port is coupled to a lumen of irrigation supply tubing.

13. A container, valve, and tube set arranged and configured to couple to an endoscope for use in an endoscopic procedure, the container, valve, and tube set comprising:
a container configured to contain a fluid, the container having a top portion and a bottom portion, the container having a port in fluid communication with the bottom portion;
fluid supply tubing including a first end, a second end, and a lumen extending therethrough, wherein the first end is in fluid communication with the port of the container and the second end is positioned external to the container;
a valve including a valve body defining:
a first port configured to receive fluid from a container via the fluid supply tubing, wherein the first port includes a spike port adaptor that is configured to be inserted into the port of the container;
a second port configured to be in fluid communication with the first port via a first flow path;
a third port configured to be in fluid communication with the first port via a second flow path;
a passage extending between the first port, the second port, and the third port;
a plunger configured to move in the passage between:
a first position at which the first flow path is open and the second flow path is closed; and
a second position at which the first flow path and the second flow path are open; and
a biasing mechanism configured to bias the plunger to the first position;
lens wash supply tubing including a first end, a second end, and a lumen extending therethrough, wherein the first end of the lens wash supply tubing is configured to be in fluid communication with the bottom portion of the container via the second flow path when the plunger is in the second position and the second end of the lens wash supply tubing is positioned external to the container and the valve; and
irrigation supply tubing having a first end, a second end, and a lumen extending therethrough, wherein the first end of the irrigation supply tubing is configured to be in fluid communication with the bottom portion of the container via first flow path, and the second end of the irrigation supply tubing is positioned external to the container and the valve.

14. The container, valve, and tube set of claim 13, wherein the valve is configured to continually provide fluid to the irrigation supply tubing when the plunger is in either the first position or the second position.

15. The container, valve, and tube set of claim 13, further comprising gas supply tubing including a first end, a second end, and a third extending therethrough, wherein the lumen of the gas supply tubing is in operative communication with the top portion of the container and the second end of the gas supply tubing is positioned external to the container.

16. The container, valve, and tube set of claim 13, further comprising:
a first seal located along an interface between a first end of the plunger and the valve body; and
a second seal located along an interface between a second end of the plunger and the valve body.

17. The container, valve, and tube set of claim 16, wherein the second seal is configured to prevent pressurized gas from backflowing along the second flow path into the container through the first port when the plunger is in the first position.

18. A container, valve, and tube set arranged and configured to couple to an endoscope for use in an endoscopic procedure, the container, valve, and tube set comprising:
a container configured to contain a fluid, the container having a top portion and a bottom portion, the container having a port in fluid communication with the bottom portion;
a valve including:
a valve body defining:
a first port configured to receive fluid from a container via fluid supply tubing, wherein the first port includes a spike port adaptor that is configured to be inserted into the port of the container;
a second port configured to be in fluid communication with the first port via a first flow path;
a third port configured to be in fluid communication with the first port via a second flow path;
a passage extending between the first port, the second port, and the third port;
a plunger configured to move in the passage between:
a first position at which the first flow path is open and the second flow path is closed; and
a second position at which the first flow path and the second flow path are open; and
a biasing mechanism configured to bias the plunger to the first position; and
lens wash supply tubing including a first end, a second end, and a lumen extending therethrough, wherein the first end of the lens wash supply tubing is configured to be in fluid communication with the bottom portion of the container via the second flow path when the plunger is in the second position and the second end of the lens wash supply tubing is positioned external to the container and the valve;
gas supply tubing including a first end, a second end, and a lumen extending therethrough, wherein the lumen of the gas supply tubing is in operative communication with the top portion and the second end of the gas supply tubing is positioned external to the container; and
irrigation supply tubing having a first end, a second end, and a lumen extending therethrough, wherein the first end of the irrigation supply tubing is configured to be permanently in fluid communication with the bottom portion of the container via first flow path to continually supply liquid to the endoscope, and the second end of the irrigation supply tubing is positioned external to the container and the valve.

19. The container, valve, and tube set of claim 18, wherein the plunger further comprises a dual piston having a first seal at a first end of the dual piston and a second seal at a second end of the dual piston that is opposite the first end.

20. The container, valve, and tube set of claim 18, wherein the first port includes a spike port adaptor that is configured to be directly inserted into the port of the container.
